(19) United States
(12) Patent Application Publication
(10) Pub. No.: US 2004/0115719 A1
(43) Pub. Date: Jun. 17, 2004

(54) Method and apparatus for analyzing a base sequence

(76) Inventors: Koji Horio, Yokohama-shi (JP); Masao Washizu, Tokyo (JP); Osamu Kurosawa, Tokyo (JP)

Correspondence Address: LAW OFFICES OF TOWNSEND & BANTA, South Building, Suite 900, 601 Pennsylvania Ave. N.W., Washington, DC, 20004, US

(21) Appl. No.: 10/691,560

(22) Filed: Oct. 24, 2003

(30) Foreign Application Priority Data
Oct. 29, 2002 (JP) 2002-314333

(51) Int. Cl. C12Q 1/68; C12P 19/34

(52) U.S. Cl. 435/6; 435/91.2

(57) ABSTRACT

Proposed is a method comprising the steps of stretching and arranging DNA while immobilizing it on a board, cutting it into fragments sequentially from one end, analyzing the respective fragments, connecting the analyzed results for analyzing the entire base sequence of the original DNA. For practically applying this method, required is a method of efficiently recovering DNA fragments without disturbing the original base sequence.

The present invention proposes a method for analyzing a base sequence, comprising the steps of forming a thin film 3 for immobilizing a base sequence test sample on the front surface of a first board 1; stretching and immobilizing a base sequence test sample 4 on the thin film; cutting the base sequence test sample in this state into fragments by means of an enzyme; heating and vaporizing the thin film in a desired region by a heating means, to shoot the fragment 7 of the base sequence test sample in the desired region from the front surface of the first board 1, in order that the fragment 7 can be arrested on the front surface of a second board 8 disposed in opposite to the front surface of the first board 1; and analyzing the base sequence in this state.

### FIELD OF THE INVENTION

The present invention relates to a method and apparatus for analyzing the base sequence of DNA or RNA.

### BACKGROUND OF THE INVENTION

A conventional base sequence analyzer for analyzing the base sequence of a base sequence test sample such as DNA can analyze hundreds of base pairs to one thousand base pairs at a time. Therefore, in the event of analyzing a base sequence test sample far longer than such numbers of base pairs, the base sequence test sample is at first cut into fragments in an aqueous solution using a restriction enzyme, and the fragments are analyzed using said base sequence analyzer.

The method of fragmenting a base sequence test sample such as DNA in an aqueous solution using a restriction enzyme has a disadvantage that the regions the analyzed fragments of the test sample had occupied in the original test sample become unknown.

Therefore, the conventional practice is such that many different restriction enzymes are used to analyze the respective fragments, and that the analyzed results are connected to estimate the entire base sequence. However, this method has such disadvantages that the analysis per se using many different restriction enzymes requires enormous labor and cost, and, in addition, that the work of connecting analyzed results for estimating the entire sequence also requires enormous labor. The disadvantages become more remarkable when a base sequence test sample such as DNA is longer.

To overcome the disadvantages, for example, the invention of Japanese Patent No. 3282679 proposes a method comprising the steps of stretching, arranging and immobilizing DNA on a board, cutting it sequentially from one end into fragments each consisting of hundreds of base pairs to one thousand base pairs, recovering them, analyzing the respective fragments, and connecting the analyzed results, for analyzing the entire base sequence of the original DNA.

For practical application of this method, required is a method for efficiently cutting a base sequence test sample such as DNA into fragments without disturbing the base sequence, and recovering the fragments.

### SUMMARY OF THE INVENTION

The present invention provides methods and apparatuses for efficiently cutting and recovering a base sequence test sample such as DNA or RNA.

The subject matter of claim 1 proposes a method for analyzing a base sequence, comprising the steps of forming a thin film for immobilizing a base sequence test sample, on the front surface of a first board; stretching and immobilizing a base sequence test sample on the thin film; cutting the base sequence test sample in this state into fragments by means of an enzyme; heating and vaporizing the thin film in a desired region by a heating means, to shoot the fragment of the base sequence test sample in the desired region from the front surface of the first board, in order that the fragment can be arrested on the front surface of a second board disposed in opposite to the front surface of the first board; and analyzing the base sequence in this state.

The subject matter of claim 2 proposes a method for analyzing a base sequence, comprising the steps of forming a thin film for immobilizing a base sequence test sample, on an ablation layer containing a material capable of being vaporized by heating, formed on the front surface of a first board; stretching and immobilizing a base sequence test sample on the ablation layer; cutting the base sequence test sample in this state into fragments by means of an enzyme; heating and vaporizing the ablation layer in a desired region by a heating means, to shoot the fragment of the base sequence test sample in the desired region from the front surface of the first board, in order that the fragment can be arrested on the front surface of a second board disposed in opposite to the front surface of the first board; and analyzing the base sequence in this state.

The subject matter of claim 3 proposes a method for analyzing a base sequence, characterized in that the base sequence analysis as set forth in claim 1 or 2 is carried out sequentially fragment by fragment from one end toward the other end of the stretched and immobilized base sequence test sample, to analyze the entire base sequence of the base sequence test sample.

The subject matter of claim 4 proposes a method for analyzing a base sequence, according to claim 1 or 2, wherein the thin film for immobilizing a base sequence test sample is a polymeric gel.

The subject matter of claim 5 proposes a method for analyzing a base sequence, according to claim 1 or 2, wherein the thin film for immobilizing a base sequence test sample has depressions and projections formed at a very small pitch.

The subject matter of claim 6 proposes a method for analyzing a base sequence, according to claim 5, wherein the material of the thin film is polymethyl methacrylate (PMMA).

The subject matter of claim 7 proposes a method for analyzing a base sequence, according to claim 5, wherein the pitch is in a range of 0.1 μm to 10 μm.

The subject matter of claim 8 proposes a method for analyzing a base sequence, according to claim 1 or 2, wherein the heating means is laser beam irradiation from the back surface of the first board.

The subject matter of claim 9 proposes a method for analyzing a base sequence, according to claim 1 or 2, wherein the heating means is an electric heater pre-formed in the first board.

The subject matter of claim 10 proposes a method for analyzing a base sequence, according to claim 2, wherein the material capable of being vaporized by heating, contained in the ablation layer, is plastic.

The subject matter of claim 11 proposes a method for analyzing a base sequence, according to claim 2, wherein in the case where laser beam irradiation from the back surface of the first board is used as the heating means, the ablation layer contains a beam-absorbable material, in addition to the material capable of being vaporized by heating.

The subject matter of claim 12 proposes a method for analyzing a base sequence, according to claim 11, wherein the beam-absorbable material is carbon.

The subject matter of claim 13 proposes a method for analyzing a base sequence, according to claim 11 or 12, wherein the beam-absorbable material is vapor-deposited between the material capable of being vaporized by heating and the first board.

The subject matter of claim 14 proposes an apparatus for analyzing a base sequence, comprising a first board having a thin film formed on its front surface for allowing a base sequence test sample to be stretched and immobilized on the thin film; a heating means for heating and vaporizing the thin film in a desired region; and a second board disposed in opposite to the front surface of the first board.

The subject matter of claim 15 proposes an apparatus for analyzing a base sequence, comprising a first board having a thin film for allowing a base sequence test sample to be stretched and immobilized, formed on an ablation layer containing a material capable of being vaporized by heating, formed on the front surface of the first board; a heating means for heating and vaporizing the ablation layer in a desired region; and a second board disposed in opposite to the front surface of the first board.

The subject matter of claim 16 proposes an apparatus for analyzing a base sequence, according to claim 14 or 15, wherein the thin film for immobilizing a base sequence test sample is a polymeric gel.

The subject matter of claim 17 proposes an apparatus for analyzing a base sequence, according to claim 14 or 15, wherein the thin film for immobilizing a base sequence test sample has depressions and projections formed at a very small pitch.

The subject matter of claim 18 proposes an apparatus for analyzing a base sequence, according to claim 17, wherein the material of the thin film is polymethyl methacrylate (PMMA).

The subject matter of claim 19 proposes an apparatus for analyzing a base sequence, according to claim 18, wherein the pitch is in a range of 0.1 μm to 10 μm.

The subject matter of claim 20 proposes an apparatus for analyzing a base sequence, according to claim 14 or 15, wherein the heating means is laser beam irradiation from the back surface of the first board.

The subject matter of claim 21 proposes an apparatus for analyzing a base sequence, according to claim 14 or 15, wherein the heating means is an electric heater pre-formed in the first board.

The subject matter of claim 22 proposes an apparatus for analyzing a base sequence, according to claim 15, wherein the material capable of being vaporized by heating, contained in the ablation layer is plastic.

The subject matter of claim 23 proposes an apparatus for analyzing a base sequence, according to claim 15, wherein in the case where laser beam irradiation from the back surface of the first board is used as the heating means, the ablation layer contains a beam-absorbable material, in addition to the material capable of being vaporized by heating.

The subject matter of claim 24 proposes an apparatus for analyzing a base sequence, according to claim 23, wherein the beam-absorbable material is carbon.

The subject matter of claim 25 proposes an apparatus for analyzing a base sequence, according to claim 23 or 24, wherein the beam-absorbable material is vapor-deposited between the material capable of being vaporized by heating and the first board.

According to this invention, a thin film for immobilizing a base sequence test sample is formed on the front surface of a first board, and a base sequence test sample such as DNA is stretched and immobilized on the thin film. In this state, an enzyme is used to cut the base sequence test sample. Therefore, after cutting, the fragments of the base sequence test sample remain immobilized on the thin film and placed in the order, in which they had been arranged in the original base sequence test sample. Furthermore, since a restriction enzyme is used for the cutting, the molecular structures remain clearly still after cutting.

Subsequently, the thin film for immobilizing a base sequence test sample or the ablation layer interposed between the thin layer and the first board is heated and vaporized in a desired region by a heating means such as laser beam irradiation or electric heater, to shoot the fragment of the base sequence test sample in the heated region, in order that the fragment can be arrested on the front surface of a second board. Therefore, the fragment of the base sequence test sample in the desired region can be reliably recovered for analyzing the base sequence.

This analysis can be carried out fragment by fragment sequentially from one end toward the other end of the base sequence test sample stretched and immobilized on the thin layer, to analyze the entire base sequence of the test sample such as DNA.

If the thin film used for immobilizing the base sequence test sample is a polymeric gel or is made of a material such as PMMA having depressions and projections formed at a very small pitch of, for example, 0.1 to 10 μm, it does not disturb the cutting by means of a restriction enzyme, and still after cutting, the immobilized state can be kept.

### BRIEF DESCRIPTION OF THE DRAWINGS

FIG. 1 is a plan view showing a state, in which a thin layer for immobilizing a base sequence test sample such as DNA is formed on the surface of a first board, DNA being stretched and immobilized on the thin film.

FIG. 2 is a partially enlarged A-A sectional view of FIG. 1.

FIG. 3 is an A-A sectional view of FIG. 1, showing a state of a certain phase.

FIG. 4 is an A-A sectional view of FIG. 1, showing a state of another phase.

### DETAILED DESCRIPTION OF THE PREFERRED EMBODIMENTS

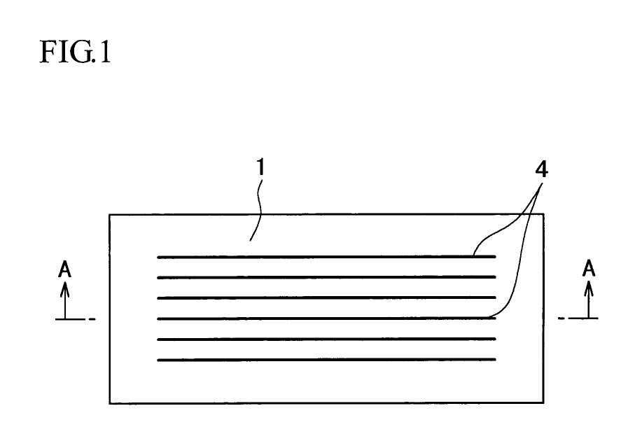

Preferred enbodiments for carrying out the invention are described below in reference to FIGS. 1 to 4.

In the drawings, symbol 1 denotes a first board, and as described later, the first board 1 is made of a light-transmitting material allowing the transmission of a laser beam. On the front surface of the first board 1, an ablation layer 2 is formed, and furthermore a thin film 3 for immobilizing a base sequence test sample is formed on the ablation layer 2. The ablation layer 2 is made of a plastic material capable of being vaporized by heating such as polyethylene, polymethyl methacrylate or Polycarbonate. The thin film 3 is formed by processing a material such as polymethyl methacrylate by such a means as photolisography or micro-molding, to have depressions and projections formed at an adequate very small pitch in a range of 0.1 to 10 μm.

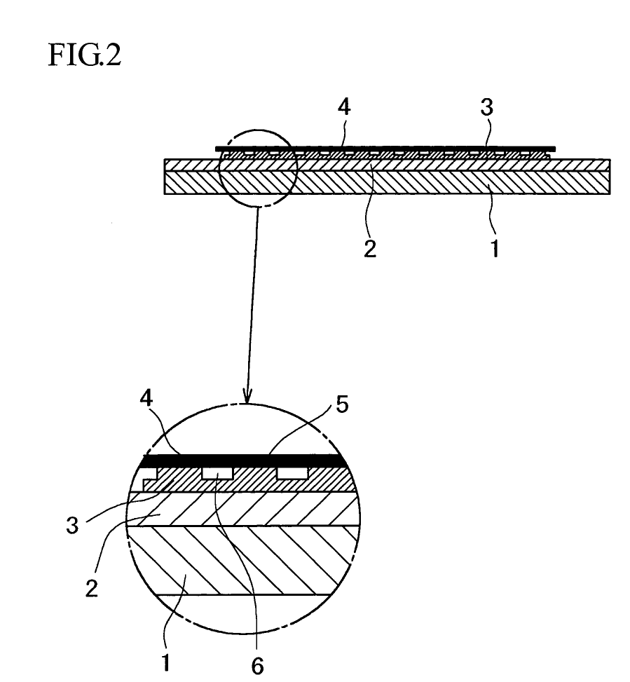

In the above-mentioned constitution, as shown in FIGS. 1 and 2, a base sequence test sample, for example, DNA 4 is stretched and immobilized on the thin film 3. For stretching the DNA 4 and immobilizing it on the thin film 3, for example as described in Japanese Patent No.3064001, the DNA in a solution can be electrostatically oriented and stretched, and the flow of the solution can be used for allowing the DNA to be deposited and immobilized. Any other adequate stretching and immobilizing method can also be used.

If a DNA-cutting enzyme (i.e. DNase) which cuts DNA regardless of the base sequence is made to act on the DNA 4 attached to the projections 5 among the depressions and projections formed at a very small pitch on the thin film 3, the enzyme does not act on the DNA 4 attached to and immobilized on the projections 5, because of the steric hindrance caused by the adsorption on the projection.

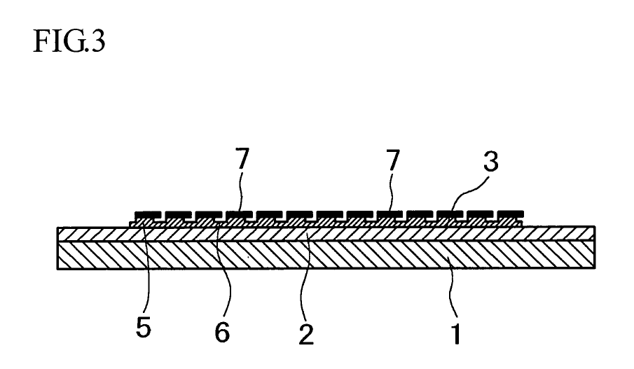

So, the DNA in the regions is not cut. However, since the enzyme acts on the DNA 4 existing over depressions 6, the DNA in the regions is cut. Therefore, as shown in FIG. 3, numerous DNA fragments 7 are obtained as supported on the projections 5 among the depressions and projections formed at a very small pitch on the thin film 3. Since the numerous DNA fragments 7 are supported on the projections of the thin film 3, they are placed in the same order as in the original DNA.

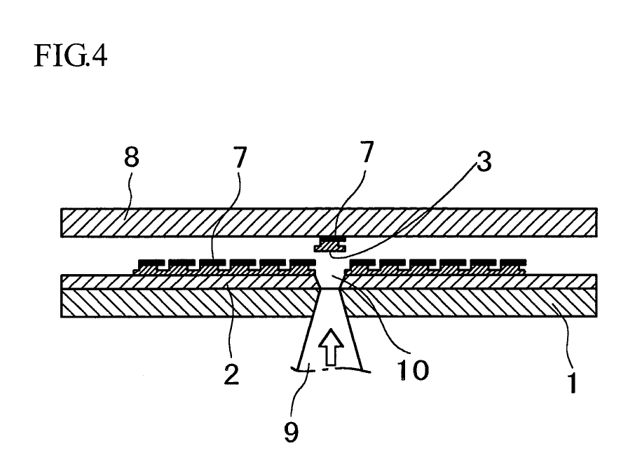

Then, as shown in FIG. 4, a second board 8 is brought to face the front surface of the first board 1, and the ablation layer 2 is irradiated with a laser beam 9 in a desired region from the back surface of the first board. As a result, the ablation layer 2 is heated and vaporized in the corresponding region 10, and its expanding force shoots the corresponding fragment of the thin layer 3 together with the DNA fragment 7, to let them adhere to the front surface of the second substrate 8.

In this invention, the DNA fragment 7 in the desired region of the original DNA 4 can be sent from the front surface of the first board 1 and arrested on the front surface of the second board 8 as described above. In this way, the DNA fragment 7 of the desired region that can be identified in the original DNA 4 can be reliably recovered for analyzing its base sequence.

The above-mentioned procedure can be carried out fragment by fragment sequentially from one end toward the other end of the DNA 4 stretched and immobilized on the thin film 3, to analyze the entire base sequence of DNA 4.

In this invention, since an enzyme is used for cutting DNA 4 or the like, the molecular structures at both the ends of each DNA fragment 7 are clearly defined. Therefore, other DNA fragments known in sequence can be easily ligated to both the ends of the DNA fragment 7, for PCR amplification using primers for the known sequences, or the DNA fragment 7 can also be easily self-cyclized for rolling circle amplification.

The second board 8 can be a sheet like the first board 1, or can also be a film. The second board 8 or the first board 1 can also be moved while the DNA fragment 7 is being arrested, in order that the entire base sequence of the original DNA is analyzed fragment by fragment sequentially.

In the above-mentioned mode, the thin film 3 for immobilizing DNA has depressions and projections formed at a very small pitch, but as another mode, a thin film composed of a polymeric gel can also be used as the thin film 3.

A polymeric gel has a network structure containing much water. So even if the network structure is used to immobilize a DNA molecule, the action of an enzyme is not disturbed since most of the DNA exists in water.

In the case where DNA is stretched and immobilized on a thin film made of a polymeric gel, cutting cannot be performed in relation with the pitch of depressions and projections unlike the thin film having depressions and projections formed at a very small pitch. In this case, it is only required to use a restriction enzyme for cutting DNA.

For example, if a restriction enzyme called a 4-base cutter capable of recognizing 4-base sequence for cutting is used, cutting occurs every 44=256 base pairs on the average in correspondence with four kinds of bases (A, T, G and C), hence every 0.34×256=87 nm, since the inter-base distance is 0.34 nm. Furthermore, if a 6-base cutter capable of recognizing 6-base sequence for cutting is used, cutting occurs every 0.34×46=1.4 μm. Thus, cutting can be carried out like the cutting of DNA into fragments with a desired length using a thin film having depressions and projections formed at a very small pitch.

In the above-mentioned embodiment, the ablation layer 2 is interposed between the first board and the thin film 3 for immobilizing a base sequence test sample, and is heated and vaporized to shoot the DNA fragment 7 corresponding to the heated region together with the corresponding fragment of the thin film 3 from the first board, in order that they are arrested on the front surface of the second board. As another embodiment, the use of the ablation layer 2 can be avoided, and the thin film 3 per se can be heated and vaporized, to shoot the DNA fragment 7.

The ablation layer 2 can contain a beam-absorbable material, in addition to the material capable of being vaporized by heating. As the beam-absorbable material, carbon used as a beam absorbent for example in laser processing can be used. If it is vapor-deposited between the material capable of being vaporized by heating and the first board, it can efficiently absorb a laser beam, to heat a plastic material or the like for efficiently heating and vaporizing the material to be vaporized by heating.

However, also in the case where laser beam irradiation is used as the heating means, if the material capable of being vaporized by heating and the wavelength of the laser beam are adequately selected, efficient heating and vaporization can be achieved even if the beam-absorbable material is not used.

In the above-mentioned embodiment, laser beam irradiation is used as the means for heating the ablation layer 2, but as another mode, an electric heater can also be disposed beforehand in the first board 1, and energized for heating the ablation layer 2 or the thin layer 3 per se in a desired region.

### INDUSTRIAL APPLICABILITY

The present invention as described above provides the following effects in analyzing the base sequence of DNA or RNA.

a. A base sequence test sample such as DNA can be efficiently cut without disturbing the base sequence, and fragments of desired regions can be reliably recovered and analyzed for analyzing the base sequence.

b. If the fragments of a base sequence test sample are recovered sequentially from one end toward the other end of the original base sequence test sample, the entire base sequence of the test sample such as DNA can be analyzed.

What is claimed is:

1. A method for analyzing a base sequence, comprising the steps of forming a thin film for immobilizing a base sequence test sample, on the front surface of a first board; stretching and immobilizing a base sequence test sample on the thin film; cutting the base sequence test sample in this state into fragments by means of an enzyme; heating and vaporizing the thin film in a desired region by a heating means, to shoot the fragment of the base sequence test sample in the desired region from the front surface of the first board, in order that the fragment can be arrested on the front surface of a second board disposed in opposite to the front surface of the first board; and analyzing the base sequence in this state.

2. A method for analyzing a base sequence, comprising the steps of forming a thin film for immobilizing a base sequence test sample, on an ablation layer containing a material capable of being vaporized by heating, formed on the front surface of a first board; stretching and immobilizing a base sequence test sample on the ablation layer; cutting the base sequence test sample in this state into fragments by means of an enzyme; heating and vaporizing the ablation layer in a desired region by a heating means, to shoot the fragment of the base sequence test sample in the desired region from the front surface of the first board, in order that the fragment can be arrested on the front surface of a second board disposed in opposite to the front surface of the first board; and analyzing the base sequence in this state.

3. A method for analyzing a base sequence, characterized in that the base sequence analysis as set forth in claim 1 or 2 is carried out sequentially fragment by fragment from one end toward the other end of the stretched and immobilized base sequence test sample, to analyze the entire base sequence of the base sequence test sample.

4. A method for analyzing a base sequence, according to claim 1 or 2, wherein the thin film for immobilizing a base sequence test sample is a polymeric gel.

5. A method for analyzing a base sequence, according to claim 1 or 2, wherein the thin film for immobilizing a base sequence test sample has depressions and projections formed at a very small pitch.

6. A method for analyzing a base sequence, according to claim 5, wherein the material of the thin film is polymethyl methacrylate (PMMA).

7. A method for analyzing a base sequence, according to claim 5, wherein the pitch is in a range of 0.1 μm to 10 μm.

8. A method for analyzing a base sequence, according to claim 1 or 2, wherein the heating means is laser beam irradiation from the back surface of the first board.

9. A method for analyzing a base sequence, according to claim 1 or 2, wherein the heating means is an electric heater pre-formed in the first board.

10. A method for analyzing a base sequence, according to claim 2, wherein the material capable of being vaporized by heating, contained in the ablation layer, is plastic.

11. A method for analyzing a base sequence, according to claim 2, wherein in the case where laser beam irradiation from the back surface of the first board is used as the heating means, the ablation layer contains a beam-absorbable material, in addition to the material capable of being vaporized by heating.

12. A method for analyzing a base sequence, according to claim 11, wherein the beam-absorbable material is carbon.

13. A method for analyzing a base sequence, according to claim 11 or 12, wherein the beam-absorbable material is vapor-deposited between the material capable of being vaporized by heating and the first board.

14. An apparatus for analyzing a base sequence, comprising a first board having a thin film formed on its front surface for allowing a base sequence test sample to be stretched and immobilized on the thin film; a heating means for heating and vaporizing the thin film in a desired region; and a second board disposed in opposite to the front surface of the first board.

15. An apparatus for analyzing a base sequence, comprising a first board having a thin film for allowing a base sequence test sample to be stretched and immobilized, formed on an ablation layer containing a material capable of being vaporized by heating, formed on the front surface of the first board; a heating means for heating and vaporizing the ablation layer in a desired region; and a second board disposed in opposite to the front surface of the first board.

16. An apparatus for analyzing a base sequence, according to claim 14 or 15, wherein the thin film for immobilizing a base sequence test sample is a polymeric gel.

17. An apparatus for analyzing a base sequence, according to claim 14 or 15, wherein the thin film for immobilizing a base sequence test sample has depressions and projections formed at a very small pitch.

18. An apparatus for analyzing a base sequence, according to claim 17, wherein the material of the thin film is polymethyl methacrylate (PMMA).

19. An apparatus for analyzing a base sequence, according to claim 17, wherein the pitch is in a range of 0.1 μm to 10 μm.

20. An apparatus for analyzing a base sequence, according to claim 14 or 15, wherein the heating means is laser beam irradiation from the back surface of the first board.

21. An apparatus for analyzing a base sequence, according to claim 14 or 15, wherein the heating means is an electric heater pre-formed in the first board.

22. An apparatus for analyzing a base sequence, according to claim 15, wherein the material capable of being vaporized by heating, contained in the ablation layer is plastic.

23. An apparatus for analyzing a base sequence, according to claim 15, wherein in the case where laser beam irradiation from the back surface of the first board is used as the heating means, the ablation layer contains a beam-absorbable material, in addition to the material capable of being vaporized by heating.

24. An apparatus for analyzing a base sequence, according to claim 23, wherein the beam-absorbable material is carbon.

25. An apparatus for analyzing a base sequence, according to claim 23 or 24, wherein the beam-absorbable material is vapor-deposited between the material capable of being vaporized by heating and the first board.
